(19) United States
(12) Patent Application Publication
(10) Pub. No.: US 2003/0060763 A1
(43) Pub. Date: Mar. 27, 2003

(54) Guide means for intraocular injection

(76) Inventors: Philip Leslie Penfold, Sydney (AU); Mark Cedric Gillies, Randwick (AU); Francis Alfred Billson, Sydney (AU)

Correspondence Address: LADAS & PARRY, 26 WEST 61ST STREET, NEW YORK, NY, 10023, US

(21) Appl. No.: 10/169,230

(22) PCT Filed: Jan. 8, 2001
(86) PCT No.: PCT/AU01/00012

(30) Foreign Application Priority Data
Jan. 6, 2000 (AU) PQ 4965

(51) Int. Cl. A61M 5/00

(52) U.S. Cl. 604/116

(57) ABSTRACT

This invention relates to the art of intraocular injection as a means of treating various conditions of the eye. In particular it relates to a plaque containing guide means for location of a needle entry point into the eye which thereby facilitates such injection. The invention also relates to a kit which includes (1) an intraocular composition containing an active compound able to treat the particular condition; (2) a syringe for dispensing the composition through a needle coupled to the syringe, the syringe having dimensions such that blockage of the needle is minimised; and (3) a plaque containing guide means for location of the needle entry points into the eye. In one form, it provides for a kit which includes (i) an anti-inflammatory steroid which is the active agent in treating the macular degeneration; (ii) a syringe need for the delivery of that steroid through a needle coupled to the syringe, and (iii) a plaque which facilitates correct positioning of the needle.

### FIELD OF THE INVENTION

This invention relates to the art of intraocular injection as a means of treating various conditions of the eye. In particular it relates to a plaque containing guide means for location of a needle entry point into the eye which thereby facilitates such injection. The invention also relates to a kit which includes (1) an intraocular composition containing an active compound able to treat the particular condition; (2) a syringe for dispensing the composition through a needle coupled to the syringe, the syringe having dimensions such that blockage of the needle is minimised; and (3) a plaque containing guide means for location of the needle entry point into the eye.

In one form, it provides for a kit which includes (i) an anti-inflammatory steroid which is the active agent in treating the macular degeneration; (ii) a syringe used for the delivery of that steroid through a needle coupled to the syringe, and (iii) a plaque which facilitates correct positioning of the needle.

### BACKGROUND OF THE INVENTION

Intraocular injection is known. For example, it is known to inject antibodies to treat intraocular infection. However, various problems may arise when using this technique. For example, if a constituent of the composition is present as sufficiently large particles, it may settle out in the vial before being drawn up into the syringe, thereby providing a non-uniform concentration of that constituent compared to its concentration in the vial.

The technique of intraocular injection itself may also cause discomfort to a patient.

It has previously been noted by our group in two earlier patent applications, details of which are discussed below, that the major cause of blindness in developed countries is a condition known as age-related macular degeneration. In this condition, the macula (which is minute area in the centre of the retina) is damaged. The macula occupies a total area of less than 1 mm². This area is especially adapted for acute and detailed vision. In the central portion of the macula, known as the fovea (which is 0.4 mm in diameter) the blood vessels, and other cells are displaced to the side, allowing light to fall onto the photosensitive layer. This is in contrast to other parts of the retina where light has to pass through several layers of tissue before arriving at the photosensitive layer.

Two of the present inventors (Billson and Penfold) obtained U.S. Pat. No. 5,770,589 the disclosure of which is incorporated herein by reference. This patent provides a method for the treatment or prophylaxis of macular degeneration in a patent and comprises administering by intravitreal injection to the patient an effective amount in depot form of an anti-inflammatory steroid which is preferably sparingly soluble in the vitreous. As set out in that document, the preferred steroid is known by its generic name as triamcinolone acetonide.

The present invention filed a further patent application (PCT/AU99/00565) directed to the prophylaxis of neovascularisation by injection of an anti-inflammatory steroid into an eye which has been identified as having a high risk of developing choroidal neovascularisation. The preferred anti-inflammatory steroid used in the method of this application is also triamcinolone acetonide.

While the results of these procedures have been encouraging, both in pilot studies and subsequent continuing clinical trials, it has been found that there is room for improvements in various aspects of the procedure.

Firstly, it has been observed that the triamcinolone acetonide may settle out on standing which may lead to inconsistencies in the amount of drug injected.

Secondly, even though a competent ophthalmologist should be able to introduce an active agent into the interior of the eye by known techniques and with minimal discomfort to the patient, this is by no means certain and some ophthalmologists may not be sufficiently confident to carry out the procedure.

It is for this reason that we have developed a kit which substantially addresses the above problems. A component of this kit is a plaque which facilitates the operation of intraocular injection.

### DISCLOSURE OF THE INVENTION

According to a first aspect of this invention, there is provided a plaque able to be positioned over an eye of a patient, said plaque having an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana.

According to a second aspect of this invention there is provided a kit for use in intraocular injection of a compound, said kit including the following:

(a) a syringe adapted to have a needle coupled thereto;

(b) a needle coupled to or to be coupled to said syringe;

(c) an intraocular composition containing an effective amount of a compound for treating a condition of an eye of a patient, said intraocular composition being contained in a container which facilitates aseptic transfer of the intraocular composition to the syringe; and

(d) a plaque able to be positioned over said eye, said plaque having an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana.

According to a third aspect of this invention there is provided a kit for use in intraocular injection of a compound, said kit including the following:

(a) a syringe adapted to have a needle coupled thereto;

(b) a needle coupled to or to be coupled to said syringe;

(c) an intraocular composition being contained within said syringe, said composition containing an effective amount of a compound for treating a condition of an eye of a patient; and

(d) a plaque able to be positioned over said eye, said plaque having an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana.

According to a fourth aspect of this invention there is provided a method of guiding a needle into the interior of an eye of a patient, said method comprising the steps of (a) positioning a plaque over the anterior surface of said eye, wherein said plaque has an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana and (b) introducing the needle through the guide means until the end of the needle is positioned in the interior of the eye.

According to a fifth aspect of this invention, there is provided a method of administering an intraocular composition containing an effective amount of a compound for treating a condition of an eye of a patient, said method comprising the steps of (a) positioning a plaque over the anterior surface of said eye, wherein said plaque has an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana; (b) introducing the needle coupled to a syringe containing said composition through the guide means until the end of the needle is positioned in the interior of the eye; and (c) expelling the contents of the syringe into the interior of the eye.

A component of the kit of this invention is a plaque which facilitates the choice of a suitable entry point for injection into the eye. As described in our previous applications, the intraocular composition should be introduced through the pars plana. The plaque generally has a profile corresponding to the convex shape of the anterior surface of the eye.

Suitably, the plaque is made of transparent or translucent material having a degree of rigidity to make it flexible to a range of corncal surfaces.

Suitably, guide means are provided on the plaque which, when placed over the eye, provide one or more entry points such that the operator can choose the optimal entry point depending on the characteristics of the eye being treated. Guide means may be, for example, apertures in the plaque. The guide means may be placed on one of the retaining means which are described below. Alternatively they may be placed on the plaque itself or on a separate projection.

The guide means are placed at distances from a position on the plaque which corresponds substantially to the centre of the iris when the plaque is placed on the eye to accommodate varying eye sizes and eye volumes. In practice, the plaque is positioned substantially over the centre of the iris. Thus, the plaque may be placed over the eye and positioned such that penetration of the eye by the needle is chosen by the clinician. The positioning of the plaque may be aided for example by a ring on the plaque showing the border between the iris and the sclera.

The length of the guide means, that is the thickness of the plaque in the region containing the guide means, is typically sufficient to allow the needle to pass therethrough and penetrate the eye to a suitable depth. In addition, the cross section of the guide means is typically such that lateral movement of a needle passing through the guide means is minimised. In other words, a needle is able to travel through the guide means and not be substantially laterally displaced.

Additionally, the plaque itself may be equipped with a stop means which regulates the depth of penetration of the eye by the needle. This may be in addition to or alternative to the scale and/or stop means on the needle mentioned above.

Preferably, the plaque has on the surface a small projection with aids the operator in holding said plaque on the eye.

Suitably, a pair of opposed retaining means directed and dimensioned to ensure retraction to the eyelids when the plaque is placed over the eye.

The main advantages of the plaque described herein are that it immobilises both the eye and eyelids. It also prevents indentation of the surface of the eye by the penetrating needle. Further, it allows correct angle of attack by the needle; suitable distance from the limbus; and suitable depth of penetration by the needle.

Conditions of the eye to which this kit is applicable are any conditions treatable by intraocular injection (including intravitreal, subtenon and orbital floor), for example a variety of exudative, ocdematous and inflammatory retinopathies including macular degeneration, diabetic retinopathy, diabetic macular oedema, cystoid macular oedema, uveitis, endophalmitis, retinal veno-occlusive disease, proliferative vitreo retinopathy and iritis; and as an adjunct to treatment such as photo-dynamic therapy which is therapeutic for macular degeneration. Further, the methods of this invention are applicable to the aphakic eye where injection may reduce the risk of after-cataract.

Examples of compounds which may be used in intraocular injection, are as follows: anti-inflammatory steroids, non-steroidal anti-inflammatory agents, metalloproteinase inhibitors, anti-angiogenic agents, antioxidants, anti-cytokine agents such as neutralising antibodies, anti-sense RNA, gene transfer vectors, anti-virals, anti-fungals, antibiotics, anti-proliferative agents, anti-metabolites, tyrosine kinase inhibitors, and calcium channel blockers.

In the case of macular degeneration, preferred steroids include 11-substituted-16α,17α-substituted methylenedioxy steroids as disclosed in our above-mentioned patent applications. The most preferred steroid is triamcinolone acetonide. Other suitable steroids may be flucinolone acetonide and anecortave acetate.

The syringe used for dispensing the active in the practice of this invention has a barrel and plunger bore of sufficiently small cross section such that application of pressure to the plunger by the operator is effective in minimising blockage of the needle by a constituent (or constituents) of the intraocular composition. Generally, the operator must use the composition which is supplied by the manufacturer and therefore has no control over the consistency of the composition. The syringe is more analogous to the type of syringe used in gas chromatography rather than the type of syringe used generally in medical practice where the cross section of the barrel and plunger are for in excess of the cross section of the needle.

Suitably, the cross section of the barrel bore is minimised so as to be able to deliver 0.1 mL or other such volume deemed necessary by the treating physician and which, but its relationship to this volume, provides optimal leverage when pressure is applied to the plunger of the syringe by the physician. For example, a syringe with a circular cross section having a diameter of 2 mm would have a length of travel of approximately 32 mm. Clearly, those skilled in the art of manufacturing syringes would be able to make a syringe with a cross section of the above order of magnitude while being able to accommodate a volume of 0.1 mL or other volume of this order chosen by the physician.

It is preferred that the needle which is to be introduced into the eye is in the range of 25 to 30 gauge. Preferably, a 27 gauge needle is used.

Optionally, the needle may be equipped with a means for indicating the distance it has penetrated into the eye. This may be in the form of a scale which is able to indicate such a distance and/or a stop means whereby the treating physician is able to predetermine the length of penetration of the needle into the eye.

The container of the second aspect of the invention containing the intraocular composition which is to be transferred into the syringe, is suitably a vial, capsule, or any other such suitable container which facilitates aseptic handling and preparation of the composition.

In compiling the kit of the third aspect of this invention, the first step is preparation of the syringe containing active which will treat the particular condition. Suitably, the composition is drawn up into the syringe (to be described below) in an amount all of which, or substantially all of which, is to be injected. In drawing up the active into the syringe it is desirable that the active in the vessel from which it is to be drawn is uniformly dispersed, thereby providing a composition in the syringe which is substantially identical in the distribution of concentration of constituents with that in the vessel from which it is drawn. Any suitable methods for maintaining homogeneous mixing may be used. For example, a magnetic stirrer or ultrasonic vibrator may be employed to achieve uniform mixing. Since the contents of the syringe are destined to be injected into the eye of a patient, it follows that the contents must be sterile. Therefore, sterility is maintained either by drawing the contents into the syringe under sterile conditions or sterilising after the contents have been drawn into the syringe by methods known in the art, for example by irradiation.

Preferably, the step of drawing up the active into the syringe is accomplished under an atmosphere of nitrogen.

As is well known in this art, the eye is prepared for injection by use of a suitable antiseptic agent, for example betadine, chlorhexidine or povidone iodine. In addition, the eye is also suitably anaesthetised by any opthalmically effective anaesthetic agent well known in the art.

It is also preferable that the surface which comes in contact with the eye has thereon a suitable, ophthalmological-grade lubricant, for example 1% hydroxymethylcellulose.

In addition, the plaque may be gas sterillised prior to placement within the kit.

### BRIEF DESCRIPTION OF THE DRAWINGS

FIG. 1 is an illustration of the type of syringe suitable for use in this invention;

FIG. 2 is an illustration of a plaque used in this invention;

FIG. 3 is an illustration of the plaque of FIG. 2 shown in profile;

FIG. 4 is an illustration of the plaque in position over the eye with a needle being introduced through one of the guide means;

FIG. 5 is an illustration of a variant of the plaque used in this invention; and

FIG. 6 is an illustration of the plaque of FIG. 5 shown in profile.

### DETAILED DESCRIPTION OF THE PREFERRED EMBODIMENT

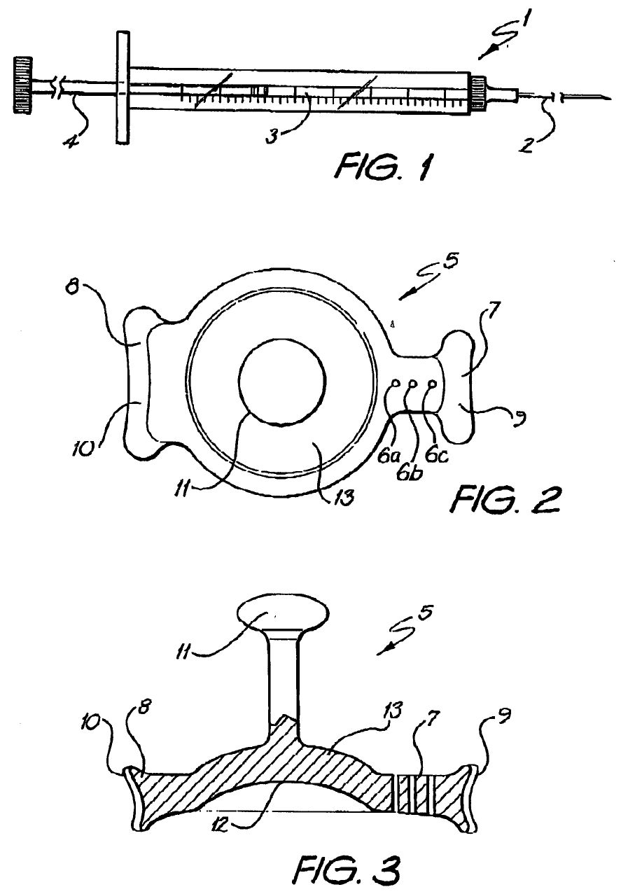

Referring to the Figures, in particular FIG. 1, a syringe for use in this invention is shown as 1. A needle 2 of narrow gauge, for example 27 gauge is coupled to the syringe by any common coupling means, for example a Luer Lock. The barrel bore 3 of the syringe is of a suitably small cross section such that application of minimal pressure to the plunger in the bore by the operator will prevent any crystals or particles blocking needle 2.

Reference numeral 5 generally shows a plaque for use in this invention. The plaque consists of guide means 6a, 6b, and 6c which are apertures formed in flange 7. The plaque has a similarly shaped flange 8 diametrically opposed. Flanges 9 and 10 formed at right angles to flanges 7 and 8 in an outward direction in relation to the eye when so placed aid in retracting the eyelids and keeping them from closing.

The patient's eye is prepared for injection by application of a suitable anaesthetic agent and a suitable antiseptic agent.

Positioning of the plaque is facilitated by a small projection 11 which assists the operator holding the plaque on the surface of the eye. The surface 12 which contacts the eye is concave relative to the convex shape of the anterior surface of the eye.

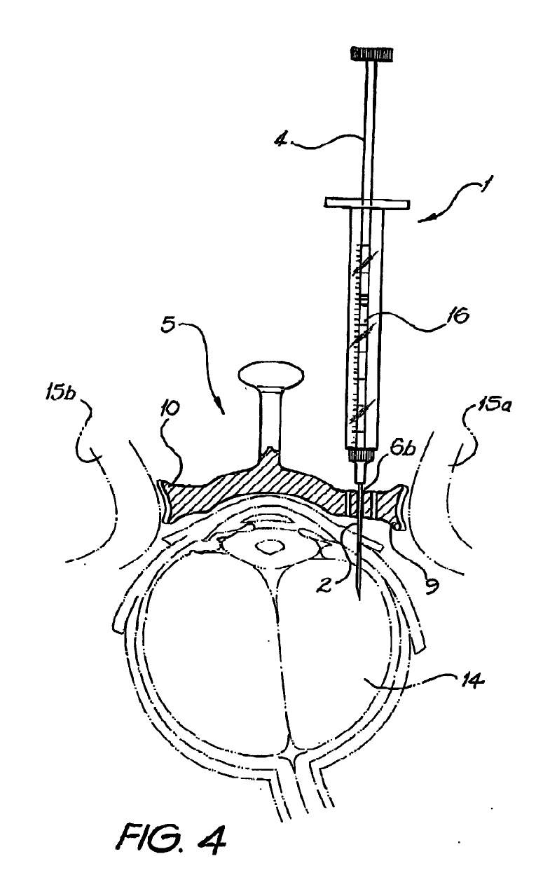

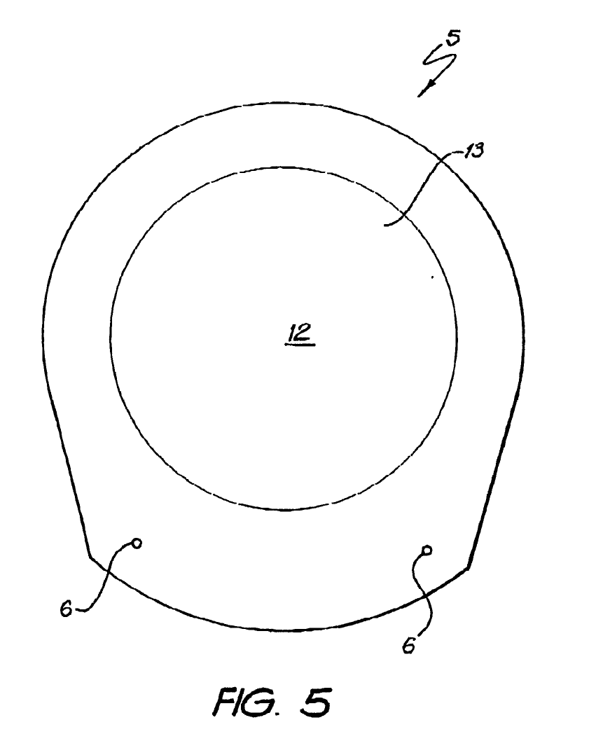

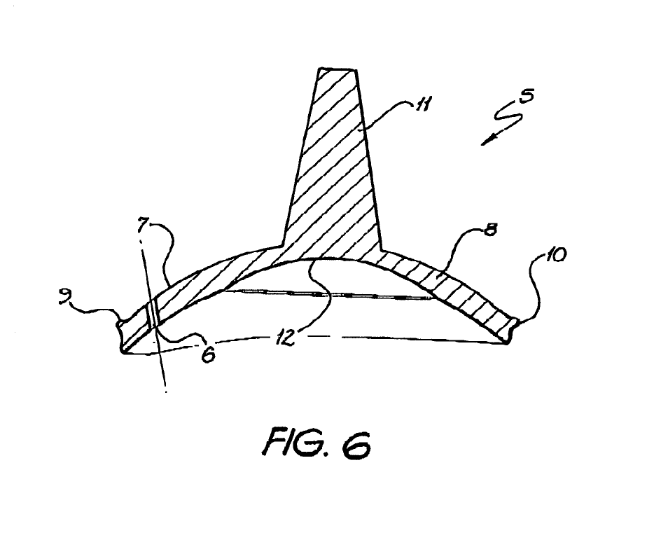

FIG. 3 generally shows the plaque 5 in cross-section thereby illustrating the concavity in the area of surface 12 to accommodate the anterior surface of the eye. FIG. 4 also shows the plaque 5 in cross-section placed over an eye (which is drawn in phantom). The syringe 1 with contents 16 and with needle 2 coupled thereto is positioned over plaque 5. The needle 2 is then introduced through an aperture (in the illustration, denoted as 6b), and pushed through the aperture through the anterior surface of the eye and brought to rest such that the tip of the needle is wholly within the interior of the eye. This is also facilitated by the needle connection being retained on the anterior surface of the plaque 5.

As can be seen in FIG. 3, needle 2 coupled to syringe 1 has been positioned through aperture 6b and has penetrated the eye at a position chosen by the operator as mentioned earlier. This position is preferably the pars plana. When positioned within the eye of the patient, the operator then depresses the plunger 4 to inject the contents 16 of syringe 1 into the vitreous 14 of the eye.

Flanges 9 and 10 are shown retracting eyelids 15a and 15b (the eyelids being shown in phantom).

### BEST MODES AND OTHER MODES FOR CARRYING OUT THE INVENTION

The present invention will now be described with reference to the following examples which should not be construed as limiting on the scope thereof.

### EXAMPLE 1

### Distribution of Steroid Into Syringes

Kenacort-A40 (Squibb) (40 mg/mL) is dispensed from material supplied by the manufacturer into a vessel. The suspension of steroid is continually mixed to ensure that aliquots removed have substantially the same range of concentrations of components as a completely mixed composition of steroid. The operation is carried out under sterile conditions and under an atmosphere of nitrogen by methods known in the art. A syringe having a delivery volume of 0.1 mL is introduced into the continually-mixed steroid solution and 0.1 mL is drawn up into the syringe. The needle is dried under sterile conditions, covered with a protective cap.

### EXAMPLE 2

### Complication of the Kit

The kit for use in this invention is compiled under sterile conditions and sealed. The kit consists of a syringe containing 0.1 mL of steroid solution and a plaque, an example of which is described above. Sterility of the contents of the kit is maintained and sealed, both of which may be accomplished by methods known in the art.

Finally, directions for using the kit are included in an outer container with the kit, together with an indication on the outside of the container of the storage conditions such as orientation of the kit and storage temperature.

### Industrial Applicability

It is envisaged that the invention will find application in the intravitreal administration of agents to treat a variety of exudative and inflammatory retinopathies including macular degeneration, diabetic retinopathy, cystoid macular oedema, uveitis endophalmitis, retinal veno-occlusive disease and proliferative vitreo retinopathy. Further, the kit would find application for the intravitreal administration of Kenacort A40 prior to and subsequent to photocoagulation and photodynamic laser therapy. The foregoing describes only some embodiments of the present invention and modifications obvious to those skilled in the art can be made thereto without departing from the scope of the invention.

1. A plaque able to be positioned over an eye of a patient, said plaque having an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana.

2. The plaque according to claim 1 wherein the guide means is placed at distances from a position on the plaque which position corresponds substantially to the centre of the iris when the plaque is placed on the eye.

3. The plaque according to claim 1 further comprising a projection on the outer surface of the plaque, the projection being positioned on the plaque and of sufficient dimensions such that a person using the plaque is able to hold the projection, to aid positioning of the plaque on the eye.

4. The plaque according to claim 1 further comprising a pair of opposed retaining means directed and dimensioned to ensure retraction of the eyelids when the plaque is placed over the eye.

5. The plaque according to claim 1 wherein the guide means is located in one of the retaining means.

6. The plaque according to claim 1 wherein the thickness of the plaque in the region containing the guide means is sufficient to allow the needle to pass therethrough and penetrate the eye to a suitable depth.

7. The plaque according to claim 1 having a stop means on the outer surface, positioned so that it regulates penetration of the needle into the eye.

8. A kit for use in intraocular injection of a compound, said kit including the following:
(a) a syringe adapted to have a needle coupled thereto;
(b) a needle coupled to or to be coupled to said syringe;
(c) an intraocular composition containing an effective amount of a compound for treating a condition of an eye of a patient, said intraocular composition being contained in a container which facilitates aseptic transfer of the intraocular composition to the syringe; and
(d) a plaque able to be positioned over said eye, said plaque having an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana.

9. A kit for use in intraocular injection of a compound, said kit including the following:
(a) a syringe adapted to have a needle coupled thereto;
(b) a needle coupled to or to be coupled to said syringe;
(c) an intraocular composition being contained within said syringe, said composition containing an effective amount of a compound for treating a condition of an eye of a patient; and
(d) a plaque able to be positioned over said eye, said plaque having an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana.

10. The kit according to claim 8 or claim 9 wherein the compound comprises an anti-inflammatory steroid, a non-steroidal anti-inflammatory agent, a metalloproteinase inhibitor, a anti-angiogenic agent, an antioxidant, an anti-cytokine agent, and anti-sense RNA, a gene transfer vector, an anti-viral, an anti-fungal, an antibiotic, and anti-proliferative agent, an anti-metabolite, a tyrosine kinase inhibitor, or a calcium channel blocker.

11. The kit according to claim 10 wherein the compound is 11-substituted-16α,17α-substituted methylenedioxy steroid.

12. The kit according to claim 11 wherein the steroid is triamcinolone acetonide, flucinolone acetonide or anecortave acetate.

13. The kit according to claim 8 or claim 9 wherein the syringe used for dispensing the active compound has a barrel and plunger bore of sufficiently small cross section such that application of pressure to the plunger by the operator is effective in minimising blockage of the needle by a constituent (or constituents) of the intraocular composition.

14. The kit according to claim 8 or claim 9 wherein the needle is in the range of 25 to 30 gauge.

15. The kit according to claim 14 wherein the needle is 27 gauge.

16. A method of guiding a needle into the interior of an eye of a patient, said method comprising the steps of (a) positioning a plaque over the anterior surface of said eye, wherein said plaque has an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana and (b) introducing the needle through the guide means until the end of the needle is positioned in the interior of the eye.

17. A method of administering an intraocular composition containing an effective amount of a compound for treating a condition of an eye of a patient, said method comprising the steps of (a) positioning a plaque over the anterior surface of said eye, wherein said plaque has an inner surface which, when the plaque is positioned over the eye, contacts the anterior surface of the eye, wherein the surface area of the inner surface of the plaque is generally equivalent to the surface area of the exposed surface of the eye when substantially open; and an outer surface which, when the plaque is positioned over the eye faces away from the eye; said plaque providing one or more guide means for guiding a needle into the interior of the eye, pars plana; (b) introducing the needle coupled to a syringe containing said composition through the guide means until the end of the needle is positioned in the interior of the eye; and (c) expelling the contents of the syringe into the interior of the eye.
